(19) United States
(12) Patent Application Publication
(10) Pub. No.: US 2001/0034546 A1
(43) Pub. Date: Oct. 25, 2001

(54) Wrist/hand conformal therapy component of an animate body heat exchanger

(76) Inventor: William Elkins, San Jose, CA (US)

Correspondence Address: C. Michael Zimmerman, P.O. Box 730393, San Jose, CA, 95173, US

(21) Appl. No.: 09/771,123

(22) Filed: Jan. 26, 2001

Related U.S. Application Data
(60) Provisional application No. 60/178,983, filed on Jan. 28, 2000.

(51) Int. Cl. A61F 7/00

(52) U.S. Cl. 607/111

(57) ABSTRACT

A wrist/hand therapy component of an animate body heat exchanger is described. Such component is designed to be wrapped snugly about the hand and associated wrist of a human, and has a penetration shaped to receive the thumb of a wearer.

CROSS REFERENCE TO RELATED APPLICATIONS

Some of the subject matter described and claimed in this patent application is also disclosed in provisional patent application Ser. No. 60/178,983 filed Jan. 28, 2000, the priority of which is hereby claimed.

### DISCLOSURE

### BACKGROUND OF THE INVENTION

The present invention relates to a conformal heat exchange therapy component of an animate body heat exchanger and, more particularly, to a wrist/hand conformal therapy component

It is now common to apply cold and compression to a traumatized area of a human body to facilitate healing and to prevent unwanted consequences of the trauma. In fact, the acronym RICE (Rice, Ice, Compression, and Elevation, is now used by many.

Cold packing with ice bags or the like has traditionally been used to provide deep core cooling of a body part. Elastic wraps are often applied to provide compression.

It will be appreciated that the traditional techniques are quite uncontrollable. For example, the temperature of an ice pack will, of course, change when the ice melts; and it has been shown that the application of elastic wraps and, consequently, the pressure provided by the same, varies considerably even when the wrapping is done by an experienced individual.

Because of these and other difficulties, many in the field have turned to more complicated animate body heat exchangers. Most effective animate body heat exchangers typically include two major components: (1) an external conformal therapy component for covering a body part to be subjected to heat exchange and compression; and (2) a control component for producing a flowing heat exchange fluid and a pressurized gas. The external conformal therapy component typically includes two bladders, a first one of which confines a heat exchange liquid or other medium and a second of which overlays the first and confines gas pressure to be applied to the body part to inhibit edema and to apply pressure against the heat exchange liquid bladder press it toward the body part.

A difficulty with many conformal therapy components is that sometimes a part of a portion of a body part to be treated, is not to be treated. For example, this is true in a conformal therapy component designed for the wrist/hand of a human. (It may be desired to use such a wrist/hand therapy component after surgery, to treat carpal tunnel syndrome, etc.) It may be desired not to treat the thumb. A wearer's thumb might interfere with obtaining the good thermal conductive relationship of the human hand to be treated. That is, the thumb interferes with the close fit which is desired.

### SUMMARY OF THE INVENTION

The present invention is a conformal therapy component designed to circumvent the above problem. It includes a penetration which is sized to received that part of an animate body that is not to be treated, but that is located at the portion that is to be treated. In a wrist/hand component it is sized to receive the thumb of a human hand. Although not necessary from the broad standpoint, in most situations the penetration extends all of the way through the remainder to the therapy component.

Most desirably, the penetration is not only sized to receive the desired part which is not to be treated and may interfere with the close conductive relationship desired with the rest of the body portion, it is also shaped to use such body part to aid in proper positioning of the remainder of the therapy component. In a wrist/hand arrangement in which it is the thumb of a human hand which is to be received by the penetration, the penetration is shaped not only to receive the thumb but also to fit about it to properly position the remainder of the therapy component.

Other features and advantages of the invention either will become apparent or will be described in connection with the following, more detailed description of a preferred embodiment of the invention and variations.

### BRIEF DESCRIPTION OF THE DRAWINGS

With reference to the accompanying sheet of drawing:

FIGS. 1A and 1B are isometric views of the front and back sides, respectively, of a preferred wrist/hand conformal therapy component incorporating the invention; and

FIG. 2 is a flattened out layout view of the major aspects of the therapy component of FIGS. 1.

### DETAILED DESCRIPTION OF THE PREFERRED EMBODIMENT

The following, relatively detailed description is provided to satisfy the patent statutes. It will be appreciated by those skilled in the art, though, that various changes and modifications can be made without departing from the invention.

A wrist/hand conformal heat exchange therapy component is generally referred to in the drawings by the reference numeral 11. It is one component of an animate body heat exchanger and, in this connection, such animate body heat exchanger also includes a control unit 12. Such control unit is connected to the wrist/hand component via three tubes 13, 14, and 16. Two of such tubes 13 and 14 are for circulating a temperature controlled liquid supplied by the control unit to the wrist/hand component, and one of such tubes, tube 16, is for the purpose of delivering gas pressure to such component as will be described. The three of such tubes are located within, and protected by, a sleeve 17 extending from the control unit adjacent the wrist/hand component. (This sleeve is often referred to as the “umbilical” cord since it represents the operative connection of the wrist/hand therapy component to the control unit.)

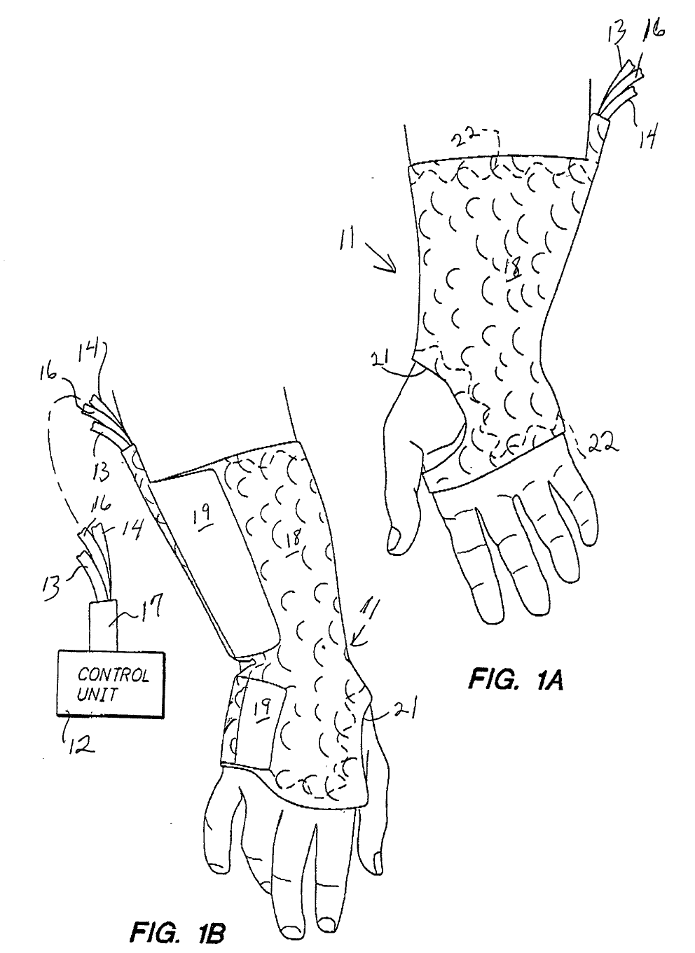

The exterior surface 18 of the wrist/hand component is covered with loop material of the “Velcro™”-type fastening mechanism. As shown in FIG. 1B, it also includes a pair of flaps 19 which have Velcro™-type hook material for interacting with the loop material. This type of fastening mechanism enables the therapy component to be wrapped snugly and wrist of an individual in close conformance thereto. (It could be said that the wrist/hand component of the invention covers a significant portion of the lower arm of an individual wearing it—the term “wrist” as used herein as meant to include such lower term “wrist” as used herein as meant to include such lower arm part.)

In keeping with the invention, the therapy component includes a penetration 21 sized to receive the thumb of the individual undergoing treatment. Although in this preferred embodiment the thumb extends all the way through the penetration from one side of the component to the other, as stated before from the broad standpoint this is not necessary—it simply is necessary for the penetration to be sized to receive that part (in this case, the thumb) that is at the body portion to be treated but is desired not be subjected to all or part of such treatment—for example, the penetration could be similar to a pocket. It also could extend through one of the bladders mentioned below but not the other.

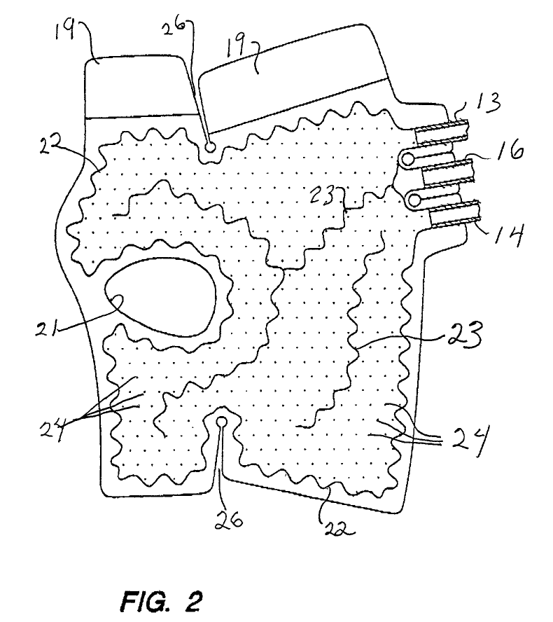

The penetration 21 is not only sized to receive the thumb, but is shaped relative to the thumb in such a way that the thumb will aid in properly positioning the wrist/hand component. Reference is made to FIG. 2 for a better showing of such shape FIG. 2 also shows flattened out, the layout for the compliant bladder which confines the heat exchange liquid and is configured to be positioned in thermally conductive relationship with the lower part of a wearer's hand and the associated wrist. The operative aspects of the wrist/hand component are a pair of bladders, the liquid bladder shown in detail in FIG. 2 and a registering gas bladder which overlays the same. Thus, he therapy component includes a pair of overlapping bladders which register with one another and are described in more detail in applicant's co-pending applications Ser. Nos. 09/127,256 and 09/173,637, the subject matters of which are incorporated herein.

The interior of the heat exchange medium bladder is defined by a curvilinear border 22. In accordance with patent application Ser. No. 09/173,637 filed originally Jul. 21, 1995, and naming the inventor herein as a co-inventor, the border 22 is formed by a plurality of curvilinear ripples which generally are significantly shorter than the length of the border portion of which each is a part. This curvilinear ripple construction, among other things, prevents eddies from forming during flow through the bladder. In this connection, while from the broad standpoint the heat exchange medium could be any appropriate substance, it is preferred that it be a temperature control liquid which i circulated by the control unit. Such liquid passes into the compliant bladder through tube 14 and is extracted therefrom through tube 13. (The temperature control agent is a flowing liquid because, among other reasons, of its thermal capacity.)

Fences or dividers 23 having the same sort of curvilinear construction as the border 22 help in defining the liquid flow. Moreover, the interior also includes a pattern of dots 24 or, in other words, a dot matrix, to disperse the liquid during its flow. Preferably, such pattern conforms to that describe in U.S. patent application Ser. No. 09/127,256 originally filed Jul. 31, 1998 and naming the instant inventor as the inventor.

In one realization of the invention, the thermal control flowing liquid was maintained at about 45° F. to cool the wrist/hand of the wearer. It was a 20 percent propylene glycol solution of distilled or de-ionized water, with a small amount of a wetting agent to break surface tension and an antifungicide such as iodine. The flow rate was about 50-60 lbs./hr. with a liquid pressure of between about 5 and 20 psig. The control unit used in such realization included an ice bath surrounding a container through which the liquid was circulated after being returned from the heat exchange bladder of the wrist/hand therapy component. (It should be noted that this control unit was capable of supplying liquid at other controlled temperatures.)

As mentioned previously, a gas pressure bladder registers with and overlays the liquid bladder. (In the embodiment shown, the gas pressure bladder is behind the liquid bladder and is not visible in detail.)

Gas pressure is introduced into the bladder via gas pressure tube 16. While any gas or other compression fluid could be used, most simply air is used and it is provided in a cyclic fashion with a cyclic pressure between peaks of about 0.25 psig and 0.15 psig, with a cycle about every two minutes from peak to peak.

The gas bladder does not include the flaps 19. The pattern of dots 24 also are nor included in the interior of the pressure bladder. However, the fences 23 may in order to prevent such 22 gas bladder from “ballooning” in accordance with the patent application filed by the inventor hereof on Jan. 16, 2001, entitled “A THERAPY COMPONENT OF AN ANIMATE BODY HEAT EXCHANGER AND METHOD OF MANUFACTURING SUCH COMPONENT.” Suitable cutouts and slits are provided to facilitate wrapping about the wrist/hand to be treated in accordance with the showings in FIGS. 1A and 1B. In this connection, the shape of the component including its compliant bladders is selected to closely fit the wrist/hand of a human. It is to be noted that this close fit results in the temperature control bladder being positioned in thermally conductive relationship with the wrist of a human, as well as with the hand of such human. The thumb penetration extends through the bladders even though, as shown in FIG. 2, the border for the flowing liquid and gas pressure itself does not circumscribe the penetration.

As mentioned at the beginning of the detailed description, applicant is not limited to the specific embodiment and variations described above. They are exemplary, rather than exhaustive. The claims, their equivalents and their equivalent language define the scope of protection.

1. A conformal therapy component of an animate body heat exchanger for applying a desired treatment to a portion of an animate body, said component comprising the combination of:
A. a compliant bladder for confining a heat exchange medium, which bladder is configured to be positioned in thermally conductive relationship with a portion of an animate body; and
B. a penetration in said component sized to receive a part of an animate body at said portion, it is desired not be subjected to all or part of said treatment.

2. The conformal therapy component of claim 1 wherein said penetration extends through said bladder for confining a heat exchange medium.

3. The conformal therapy component of claim 1 further including as part of said combination, a compliant gas pressure bladder overlaying at least a portion of the heat exchange medium bladder to direct gas pressure against said heat exchange medium bladder and press the same toward said body portion.

4. The conformal therapy component of claim 1 in which said penetration extends all of the way through said component.

5. The conformal therapy component of claim 4 in which said penetration is shaped to fit said part and thereby aid in positioning said component at said portion of an animate body.

6. The conformal therapy component of claim 1 designed for applying a desired treatment to a human hand in which said compliant bladder is configured to be positioned in thermally conductive relationship with the lower portion of said hand, and said penetration is sized to receive the thumb of a human hand.

7. The conformal therapy component of claim 6 in which said compliant bladder is also configured to be positioned in thermally conductive relationship with the wrist of a human associated with said human hand.

8. A conformal therapy component of an animate body heat exchanger for applying a desired treatment to a portion of an animate body, said component comprising the combination of:
A. a compliant bladder for confining a heat exchange medium, which bladder is configured to be positioned in thermally conductive relationship with a portion of an animate body;
a compliant gas pressure bladder overlaying at least a portion of the heat exchange medium bladder to direct gas pressure against said heat exchange medium bladder and press the same toward said body portion; and
C. a penetration in said component extending through both of said compliant bladders, which penetration is sized to receive a part of an animate body at said portion, it is desired not be subjected to all or part of said treatment.

9. The conformal therapy component of claim 8 wherein said compliant bladder for confining a heat exchange medium is configured to be positioned in thermally conductive relationship with both the lower portion of a hand of a human and the wrist associated with said hand, and said penetration is sized to receive the thumb of said hand.

10. The conformal therapy component of claim 8 wherein said penetration is shaped to fit said part and thereby aid in positioning said component at said portion of an animate body.
